(19) United States
(12) Patent Application Publication
(10) Pub. No.: US 2010/0166848 A1
(43) Pub. Date: Jul. 1, 2010

(54) LIPOSOMAL FORMULATIONS FOR TREATING CANCER

(76) Inventors: Joseph Ciccolini, Auriol (FR); Raphaelle Fanciullino, Marseille (FR)

Correspondence Address: OCCHIUTI ROHLICEK & TSAO, LLP, 10 FAWCETT STREET, CAMBRIDGE, MA, 02138, US

(21) Appl. No.: 12/676,013

(22) PCT Filed: Sep. 11, 2008
(86) PCT No.: PCT/EP08/62103

§ 371 Date: Mar. 2, 2010

Related U.S. Application Data
(60) Provisional application No. 60/960,017, filed on Sep. 11, 2007.

(51) Int. Cl. A61K 9/127; A61K 31/513; A61P 35/00

(52) U.S. Cl. 424/450; 514/263.3

(57) ABSTRACT

The invention provides a pharmaceutical composition comprising liposomes which coencapsulate 5-fluorouracil (5-FU) and 2′-deoxyinosine (d-Ino). In a preferred embodiment, the liposomes comprise phosphatidylcholine (PC), phosphatidyl-glycol (PG) and sterol, preferably in a molar ratio PC/PG/sterol of 20-80/10-60/15-60. The invention further provides a method for preparing liposomes which coencapsulate 5-fluorouracil (5-FU) and 2′-deoxyinosine (d-Ino), and the use thereof for the manufacture of a medicament for treating cancer in a patient.

The invention relates to liposomal compositions comprising 5-fluorouracil (5-FU), which are useful in cancer chemotherapy.

### BACKGROUND OF THE INVENTION

Despite the fact that 5-fluorouracil (5-FU) has been in use for half a century, it remains the gold standard for chemotherapy of colorectal cancer, the third cause of death due to cancer worldwide. With a mere 20% response rate when used as monotherapy, numerous attempts have been made to improve its therapeutic index, both at the bench and at the bedside. 5-FU was rationally designed to target thymidylate synthase (TS), an enzyme that is essential for DNA synthesis and cell proliferation; however, the biochemical mechanisms responsible for its antitumor properties are complex and actually require anabolism of this prodrug into specific 5-FU nucleotides within cancer cells. Several enzymes involved in its metabolic activation eventually lead to the formation of active cytotoxic nucleotides or deoxynucleotides (Boyer et al, 2004). However, the major mechanism for 5-FU cytotoxicity is the formation of competitive 5-fluoro-2-deoxyuridine-5-monophosphate (FdUMP), thereby inhibiting TS activity with subsequent depletion of intracellular thymidine, suppression of DNA synthesis, and ultimately, apoptosis induction (Pinedo et al, 1988; Diasio et al, 1989; Langley et al, 2003). Overexpression of TS has been demonstrated to be associated with 5-FU resistance in patients with colorectal cancer (Johnston et al, 1995; Peters et al, 2002). In this respect, controlling the pattern of 5-FU activation, preferentially towards inhibiting TS FdUMP synthesis, is a major goal for optimizing its anticancer efficacy.

The key-enzyme in the process of yielding intratumoral FdUMP is thymidine phosphorylase (TP), the rate-limiting enzyme in the activation of 5-FU via the DNA pathway (Ciccolini et al, 2001, De Bruin et al, 2004). Several attempts to boost tumoral TP levels have been published in an effort to improve cell sensitivity to 5-FU or oral 5-FU (capecitabine), by using either “Suicide Gene” strategies (Schwartz et al, 1995a and 1995b; Evrard et al, 1999; Ciccolini et al, 2001), co-treating tumor cells with modulators such as IFN, taxoïd drugs, Mitomycine C, or with radiotherapy (Ciccolini et al, 2004, Blanquicett et al 2005). Among the numerous compounds tested as putative modulators, 2′-deoxyinosine (d-Ino), is a non-toxic precursor of the TP cofactor, deoxyribose 1-phosphate, that has been shown to enhance 5-FU's antiproliferative activity in several in vitro and in vivo models, when either used alone or combined with gene therapy strategies targeting TP (Ciccolini et al, 2000a, 2000b, 2001, Fanciullino et al, 2006). So far, extensive erythrocytic metabolism and a failure to improve its pharmacokinetic profile have prevented d-Ino from being considered clinically, as a possible modulator of 5-FU. A first encapsulated formulation of d-Ino alone (d-InoL) was designed to spare it from erythrocytic clearance (Fanciullino et al, 2005).

The inventors have now succeeded in developing a double-liposomal formulation encapsulating both 5-FU and its modulator, d-Ino, which enhances the therapeutic index of 5-FU.

### SUMMARY OF THE INVENTION

The invention provides a pharmaceutical composition comprising liposomes which coencapsulate 5-fluorouracil (5-FU) and 2′-deoxyinosine (d-Ino).

In a preferred embodiment, the liposomes comprise phosphatidylcholine (PC), phosphatidylglycol (PG) and sterol, preferably in a molar ratio PC/PG/sterol of 20-80/10-60/15-60, more preferably a molar ratio PC/PG/sterol of 30-50/20-30/20-40, still more preferably a molar ratio PC/PG/sterol of 35-45/24-28/24-38.

Advantageously the sterol may be cholesterol.

The composition may further comprise polyethylene glycol (PEG) out of the liposome membrane.

Preferably the liposomes comprise phosphatidylcholine (PC), phosphatidylglycol (PG), sterol and PEG in a molar ratio PC/PG/sterol/PEG of 20-80/10-60/15-60/0.30-1.5.

In a most preferred embodiment, the liposomes comprise a molar ratio of PC/PG/cholesterol/PEG of about 40/26/33/0.68.

The invention further provides a method for preparing liposomes which coencapsulate 5-fluorouracil (5-FU) and 2′-deoxyinosine (d-Ino), and the use thereof for the manufacture of a medicament for treating cancer in a patient.

The medicament may be particularly useful in patients who show chemoresistance to 5-FU or to another fluoropyrimidine drug.

### DETAILED DESCRIPTION OF THE INVENTION

Drug resistance is a major cause of treatment failure in cancer chemotherapy, including that with the extensively-prescribed antimetabolite, 5-FU. The inventors propose to reverse 5-FU resistance by using a double punch strategy: combining 5-FU with a biochemical modulator to improve its tumoral activation and encapsulating both these agents in one same stealth liposome.

Experiments carried out in the highly resistant, canonical SW620 human colorectal model showed an up to 80% sensitization to 5-FU when these cells were treated with the liposomal formulation of the invention. Results with this formulation demonstrated 30% higher tumoral drug uptake, better activation with increased active metabolites including critical-FdUMP, superior inhibition (98%) of tumor thymidylate synthase, and subsequently, higher induction of both early and late apoptosis. Drug monitoring showed that higher and sustained exposure was achieved in rats treated with liposomal formulation. When examined in a xenograft animal model, the dual-agent liposomal formulation of the invention caused a 74% reduction in tumor size with a mean doubling in survival time, whereas standard 5-FU failed to both exhibit significant antiproliferative activity and increase the lifespan of tumor-bearing mice.

Suitable liposomes for use in this invention include large unilamellar vesicles (LUVs), multilamellar vesicles (MLVs), small unilamellar vesicles (SUVs) and interdigitating fusion liposomes, most preferably they are small unilamellar vesicles (SUVs).

Preferably, liposomes of the invention contain a sterol, such as cholesterol. The presence of 30 mol % of cholesterol significantly modifies the phase transition characteristics of the lipids and incorporating cholesterol permit membrane-rigidifying.

Liposomes of the invention may also contain therapeutic lipids, which examples include ether lipids, phosphatidic acid, phosphonates, ceramide and ceramide analogs, sphingosine and sphingosine analogs and serine-containing lipids.

Liposomes may also be prepared with surface stabilizing hydrophilic polymer-lipid such as polyethylene glycol-DSPE, to enhance circulation longevity. The incorporation of negatively charged lipids such as phosphatidylglycerol (PG) may also be added to liposome formulations to increase the circulation longevity of the carrier.

Various methods may be utilized to encapsulate the active agents (i.e. 5-fluorouracil (5-FU) and 2′-deoxyinosine (d-Ino)) in liposomes.

“Encapsulation” includes covalent or non-covalent association of an agent with the lipid-based delivery vehicle. For example, this can be by interaction of the agent with the outer layer or layers of the liposome or entrapment of an agent within the liposome, equilibrium being achieved between different portions of the liposome. Thus encapsulation of an agent can be by association of the agent by interaction with the bilayer of the liposomes through covalent or non-covalent interaction with the lipid components or entrapment in the aqueous interior of the liposome, or in equilibrium between the internal aqueous phase and the bilayer. “Loading” refers to the act of encapsulating one or more agents into a delivery vehicle. The therapeutic agents may be loaded into liposomes using both passive and active loading methods. Passive methods of encapsulating active agents in liposomes involve encapsulating the agent during the preparation of the liposomes. This includes a passive entrapment method described by Bangham, et al. (J. Mol. Biol. (1965) 12: 238). This technique results in the formation of multilamellar vesicles (MLYs) that can be converted to large unilamellar vesicles (LUVs) or small unilamellar vesicles (SUVs) upon extrusion. Additional suitable methods of passive encapsulation include an ether injection technique described by Deamer and Bangham (1976) and the Reverse Phase Evaporation technique as described by Szoka and Paphadjopoulos (1978).

Active methods of encapsulation include the pH gradient loading technique described in U.S. Pat. Nos. 5,616,341, 5,736,155 and 5,785,987 and active metal-loading. A preferred method of pH gradient loading is the citrate-base loading method utilizing citrate as the internal buffer at a pH of 4.0 and a neutral exterior buffer.

Other methods employed to establish and maintain a pH gradient across a liposome involve the use of an ionophore that can insert into the liposome membrane and transportions across membranes in exchange for protons (see U.S. Pat. No. 5,837,282). A recent technique utilizing transition metals to drive the uptake of drugs into liposomes via complexation in the absence of an ionophore may also be used. This technique relies on the formation of a drug-metal complex rather than the establishment of a pH gradient to drive uptake of drug.

Metal-based active loading typically uses liposomes with passively encapsulated metal ions (with or without passively loaded therapeutic agents). Various salts of metal ions are used, presuming that the salt is pharmaceutically acceptable and soluble in an aqueous solutions. Actively loaded agents are selected based on being capable of forming a complex with a metal ion and thus being retained when so complexed within the liposome, yet capable of loading into a liposome when not complexed to metal ions. Agents that are capable of coordinating with a metal typically comprise coordination sites such as amines, carbonyl groups, ethers, ketones, acyl groups, acetylenes, olefins, thiols, hydroxyl or halide groups or other suitable groups capable of donating electrons to the metal ion thereby forming a complex with the metal ion.

Passive and active methods of entrapment may also be coupled in order to prepare a liposome formulation according to the invention.

The ratio of 5-fluorouracil (5-FU) to 2′-deoxyinosine (d-Ino) in each liposome is generally about 1:1, or 1:1-1,5 in molar.

In a preferred embodiment, the method for preparing liposomes which coencapsulate 5-fluorouracil (5-FU) and 2′-deoxyinosine (d-Ino) comprises the steps consisting of

(a) dissolving a lipid mixture comprising phosphatidylcholine (PC), phosphatidylglycol (PG) and sterol in an alcoholic solvent, and removing the solvent to form a lipid film;
(b) hydrating the lipid film with a solution containing 5-fluorouracil and 2′-deoxyinosine, to form a lipid dispersion;
(c) forming Multilamellar vesicles (MLVs) by mixing the lipid dispersion;
(d) obtaining Small unilamellar vesicles (SUVs) therefrom, by sonication.

In the above method, the solution containing 5-fluorouracil and 2′-deoxyinosine preferably contains a carbonate buffer pH 7.4.

In a particular embodiment, the liposomes further encapsulate folinic acid.

Folinic acid is a 5-formyl derivative of tetrahydrofolic acid, and is also known as leucovorin.

Liposomes which coencapsulate 5-Fu, d-Ino, and folinic acid may be obtained by the above method, wherein the solution in step (b) further comprises folinic acid.

The liposomes which coencapsulate 5-Fu, d-Ino, and folinic acid may also comprise phosphatidylcholine (PC), phosphatidylglycol (PG) and sterol in a molar ratio PC/PG/sterol of 20-80/10-60/15-60. The liposomes comprise preferably a molar ratio of PC/PG/cholesterol/PEG of about 26/16.7/21/0.44.

Preferably liposomes according to the invention have a mean diameter of less than 300 nm. Most preferably liposomes have a mean diameter of less than 200 nm, preferably a mean diameter between 50 nm and 200 nm. Tumor vasculature is generally leakier than normal vasculature due to fenestrations or gaps in the endothelia. This allows the delivery vehicles of 200 nm or less in diameter to penetrate the discontinuous endothelial cell layer and underlying basement membrane surrounding the vessels supplying blood to a tumor. Selective accumulation of the delivery vehicles into tumor sites following extravasation leads to enhanced anticancer drug delivery and therapeutics effectiveness.

Preferably, the pharmaceutical compositions of the present invention are administered parenterally, i.e. intraarterially, intravenously, intraperitoneally, subcutaneously, or intramuscularly. More preferably, the pharmaceutical compositions are administered intravenously or intraperitoneally by a bolus injection. For example, see Rahman, et al., U.S. Pat. No. 3,993,754; Sears, U.S. Pat. No. 4,145,410; Papahadjopoulos, et al., U.S. Pat. No. 4,235,871; Schneider, U.S. Pat. No. 4,224,179; Lenk, et al., U.S. Pat. No. 4,522,803; and Fountain, et al., U.S. Pat. No. 4,588,578.

In other methods, the pharmaceutical compositions of the present invention can be contacted with the target tissue by direct application of the composition to the tissue.

Pharmaceutical compositions may further comprise pharmaceutically acceptable excipients, including water, buffered water, 0.9% saline, 0.3% glycine, 5% dextrose and the like, including glycoproteins for enhanced stability, such as albumin, lipoprotein, globulin, and the like. These compositions may be sterilized by conventional, well-known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and the like. Additionally, the delivery vehicle suspension may include lipid-protective agents which protect lipids against free-radical and lipid-peroxidative damages on storage. Lipophilic free-radical quenchers, such as alpha-tocopherol and water-soluble iron-specific chelators, such as ferrioxamine, are suitable.

The concentration of liposomes in the pharmaceutical compositions can vary widely, such as from less than about 0.05%, usually at or at least about 2-5% to as much as 10 to 30% by weight and will be selected primarily by fluid volumes, viscosities, and the like, in accordance with the particular mode of administration selected. For example, the concentration may be increased to lower the fluid load associated with treatment.

Preferably, the pharmaceutical compositions of the present invention are administered intravenously. Dosage for the delivery vehicle formulations will depend on the ratio of drug to lipid and the administrating physician's opinion based on age, weight, and condition of the patient. The combination of 5-FU and d-INO in the same liposomes makes it possible to reduce the dosage of 5-FU, compared to the dosage of 5-FU required when used alone. For example, for an intravenous infusion, a regimen of less than 300 mg/m²/day, preferably less than 150 mg/m²/day, in 250 ml 5% glucose serum in 1 h30, during 5 days or less, is proposed.

The compositions of the present invention may be administered to any mammals, especially humans.

Liposome compositions in accordance with this invention are preferably used to treat cancer, preferably solid cancers, including colorectal, breast or head and neck cancers. Advantageously the targeted cancers are drug-resistant solid cancers such as 5-FU resistant colorectal, breast or head and neck cancers overexpressing thymidylate synthase. The liposome compositions of the invention may be also useful in cancer patients who show chemoresistance to another fluoropyrimidine drug, such as capecitabine, UFT, ftorafur, or S1.

The following figures and examples are offered to illustrate but not to limit the invention.

### LEGENDS TO THE FIGURES

FIG. 1: Monitoring of d-Ino (♦) and 5-FU (□) release from the liposomal [5-FU+d-Ino]-L form. d-Ino and 5-FU concentrations were measured at 248 nm. Bars: SD.

FIG. 2: Modulation of 5-FU (⋄) cytotoxicity by free d-Ino (▪) or as the co-encapsulated [5-FU+d-Ino]-L form (▴). Cells were treated for 24 h and viability was measured by MTT-testing after 48 extra-h of growth in drug-free medium. Bars: SD.

FIG. 3: Monitoring of the intratumoral activation of tritiated 5-FU alone, associated with free d-Ino, or as a liposomal [5-FU+d-Ino]-L combination. Data are from one representative experiment. FUH2: dihydrofluorouracil, FURd: fluorouridine, FUMP: fluorouridine monophosphate, FUDP: fluorouridine diphosphate, FUTP: fluorouridine triphosphate, FdURd: fluorodeoxyuridine, FdUMP: fluorodeoxyuridine monophosphate, FdUDP: fluorodeoxyuridine diphosphate, FdUTP: fluorodeoxyuridine triphosphate.

FIG. 4: Potentiation of TS inhibition by 5-FU alone, combined with free d-Ino or with the liposomal |5-FU+d-Ino]-L association. Cells were exposed for 24 h to 100 μM 5-FU alone, in combination with 250 μM d-Ino or to the encapsulated association.

FIG. 5: Cell-death induction by 5-FU alone, combined with free d-Ino or with the liposomal |5-FU+d-Ino]-L association. Cells were exposed for 24 h to 100 μM 5-FU alone or associated with 250 μM d-Ino. Cell death was measured at 48 and 72 h after PI staining and flow-cytometry analysis.

FIG. 6: Enhancement of 5-FU-induced apoptosis. SW620 cells were exposed to 100 μM of 5-FU alone or combined with 250 μM d-Ino, either free or as a liposomal combination. Early and late apoptosis were discriminated by PI/Annexin V double staining with subsequent flow-cytometry analysis.

FIG. 7: Effects of the liposomal association [5-FU+d-Ino]-L on SW620 tumor growth in nude mice. Animals (n=5/group) were subcutaneously transplanted with SW620 tumoral cells and administered for three consecutive days, over three consecutive weeks with each of the following: carbonate (daily IP); 5-FU (50 mg/kg daily IP) alone or combined with d-Ino (120 mg/kg daily IP), or as the [5-FU+d-Ino]-L formulation (50 mg/kg+120 mg/kg respectively). Bars: SD.

FIG. 8: Kaplan-Meyer representation of tumor-bearing mice's survival treated either with 5-FU alone, 5-FU+d-Ino, or with the [5-FU+d-Ino]-L combination, as compared with untreated animals.

### EXAMPLES

### Example 1

### In Vitro and In Vivo Reversal of Resistance to 5-FU in Colorectal Cancer Cells

### Material and Methods

### Cell Lines.

All experiments were carried out in the 5-FU-resistant, human colon carcinoma cell line SW620 (a.k.a CCL227) which overexpresses TS. Cells were maintained in RPMI supplemented with 10% fetal calf serum, 5% glutamine, 10% penicillin, 10% streptomycin and 1% kanamycin in a humidified CO₂ incubator at 37° C. All experiments were performed in exponentially growing cells.

### Drugs and Chemicals.

Egg yolk phosphatidylcholine (PC), phosphatidylglycerol (PG), cholesterol (C), polyethylene glycol (PEG) covalently binded to phosphatidylethanolamine, 2′-deoxyinosine (d-Ino), 5-Fluorouracil (5-FU) and 5′ dFUR were all purchased from Sigma (St Quentin, France). Di-kalium hydrogenous phosphate (K₂HPO₄) buffer, tetrabutyl ammonium nitrate (TBAN), acetonitrile, ether and methanol came from CarboErba (Milan, Italy). Dimethyl sulfoxide, the apoptosis kit, and culture media were purchased from Euromedex (Souffelweyersheim, France). Tritiated 5-FU (12Ci/mMol) was obtained from Moraveck Biochemical (Brea, Calif., USA). All reagents were of analytical grade.

### Liposome Preparation.

Liposomes ([5-FU+d-Ino]-L) were prepared by the classic thin film method (Olson et al, 1979).

In brief, a lipid mixture composed of egg PC/PG/CHOL/PEG (molar ratios of 40:26:33:0.68) in methanol was evaporated under nitrogen in a round bottom flask to form a dried thin film. This film was then hydrated with an isotonic carbonate solution (Ph 4.2-7.4). The ratio: neutral phospholipid to cholesterol was 30% and when present, the negatively charged lipid was 10% of the neutral lipid. Multilamellar vesicles were formed by vortex mixing the lipid dispersions at room temperature. 5-FU (0.2 mM) and d-Ino (0.24 mM) were encapsulated by incubation with the lipid film for 30 min at 37-40° C. The resulting loaded PEG-liposomes were then shaken. Homogenous size distribution as SUV was achieved by 5 min 20 KHz sonication with a probe. To remove the non-encapsulated drug, the liposomal suspension was ultracentrifuged at 70,000×g at 4° C. for 16 h., and the resulting pellet was resuspended in either 10 ml of culture media or 10 mM carbonate buffer (pH 7.4), depending on its use (e.g. in vitro, or in vivo). Finally, sterile liposomes were obtained after extrusion through PVDF filters (Durapore 0.22 μm, Millipore, Molsheim, France).

### Polydispersity Study.

Diameter and particle size distribution were determined by dynamic laser light scattering using a Correlateur RTG submicron particle analyser (Sematech, Nice, France). Measurements were performed at 90° angles, at room temperature. The mean diameter of the liposomes was estimated from the volume distribution curves given by the particle analyzer.

### Encapsulation Rate and Releasing Study.

Encapsulation rates of both 5-fu and d-Ino were performed by hplc, using a previously-published method (Fanciullino et al, 2005). Liposomel release of 5-FU and d-Ino was monitored by dialysis as described elsewhere (Fanciullino et al, 2005). Sampling was performed every 30 min, up to 4 h. The total amount of 5-FU and d-Ino released was determined by UV-spectrophotometry at 248 nm (Beckman, France).

### Antiproliferative Assays.

Cells were seeded at a density of 8×10⁴ cells/well in 96-well plates. After overnight attachment, exponentially growing cells were exposed to increasing concentrations of 5-FU alone, 5-FU combined with free d-Ino, or the liposomal formulation [5-FU+d-Ino]-L, for 24 h. Next, drug was removed and the cells were allowed to grow in fresh medium for an additional 48 h. After 72 h of discontinuous exposure, cell viability was evaluated using the classic colorimetric MTT assay (Alley et al, 1988). The IC₅₀ was defined as the 5-FU concentration inhibiting 50% of cell growth.

### 5-FU Tumoral Metabolism Study.

Exponentially growing cells were exposed to 1 μM of tritiated 5-FU alone, combined with free d-Ino or as a liposomal preparation [5-FU+d-Ino]-L. After 3, 4 and 6 h exposure, cells were harvested, lysed into 70% methanol and cytosols were isolated by centrifugation (15000 rpm, 30 min) and stored at −80° C. until analysis. Separation of 5-FU and its main metabolites was achieved using a HP1090 HPLC system (Agilent, France) coupled to a Flo-One radioactive detector (Packard, France) and equipped with an RP18 column (Agilent, France), followed by elution with a K₂HPO₄-TBAN:Methanol gradient as described previously (Ciccolini et al, 2000b).

### TS Inhibition.

TS Activity was assessed as described previously (Chazal et al, 1997). Briefly, exponentially growing cells were exposed to various combinations of 100 μM of 5-FU alone, 5-FU combined with 250 μM d-Ino, or the liposomal formulation [5-FU+d-Ino]-L for 12 h. Inhibition of TS activity was evaluated at 8, 24, 48 and 72 h. Cells were then harvested and the pellet was stored at −80° C. until further analysis. TS activity was assayed following the standard Roberts' method based on tritiated H₂O release from [³H]-deoxyuridine monophosphate, in the presence of excess methylene tetrahydrofolate (Roberts et al, 1966).

### Cell Death Induction.

Cell cycle distribution was monitored after exposing the cells for 48 and 72 h to 100 μM 5-FU, 5-FU combined with 250 μM of free d-Ino, or the [5-FU+d-Ino]-L combination at the same concentrations. Cells were washed twice with PBS, trypsinized, and suspended in 70% methanol for 1 h, at 4° C. Next, they were centrifuged and immediately collected in 300 μL PBS and 80 μL of propidium iodide (PI), following the manufacturer's recommendations. Samples were analyzed with FACScan flow cytometer (Beckman Coulter, France) using Cell Quest software. The percentage of cell death was measured by detecting the sub-G0/G1 peak in PI staining (Derzynkiewicz et al, 1992).

### Apoptosis Studies.

Cells in the exponential phase were exposed to 100 μM 5-FU alone, 5-FU with 250 μM d-Ino, or the liposomal combination [5-FU+d-Ino]-L for 24, 48 and 72 hours. Cells were harvested, and early as well as late apoptotic changes were detected by simultaneous staining with Annexin and propidium iodide, using an Annexin V FITC staining kit (Sigma, St Quantin Fallavier, France). Cells were treated following the manufacturer's guidelines. FACS analysis was carried out in a FACScan flow cytometer (Becton Dickinson, Poisat, France) using the Cell Quest Software, and apoptosis measured in untreated cells was defined as 100%.

### Drug Monitoring Study.

Comparison of the drug exposure levels achieved in animals treated with the free or encapsulated 5-FU+d-Ino combination was performed. Six-weeks old male wistar rats (Charles River, Lyon, France) were kept anaesthetized using O₂/NO₂ gaz+isoflurane (TEM, Bordeaux, France) during the whole study. Body temperature was maintained at 37° C. using a warming-blanket. Animals (n=3/group) were administered by intra-peritoneal injection with 5-FU (50 mg/kg) plus d-Ino (120 mg/kg), either free or combined in the liposomal formulation. Sampling times were as following: T0, T60, T90 and T120 min. One-ml of blood were withdrawn from jugular vein on heparinized tubes, and plasma was isolated by centrifugation at 5000 g for 10 min. Samples were stored at −20° C. until analyzed. 5-FU and d-Ino plasma concentrations were determined by reverse-phase UV-HPLC as described previously, using 2-deoxyadenosine as internal standard (Fanciullino et al, 2005). Animal study was performed following animal welfare guidelines, and local animal ethics committee approval was obtained prior to starting the experiments.

### In Vivo Efficacy Studies.

The antitumor efficacy of 5-FU alone, or in association with free d-Ino, or as the liposomal [5-FU+d-Ino]-L combination was investigated in the SW620 mouse xenograft model. Mouse care was in agreement with the animal welfare guidelines of our institution, and local animal ethics committee approval was obtained prior to starting the experiments. Four-week old, female Swiss, nude mice (n=5 per group, Charles River, Lyon, France) were subcutaneously inoculated with 1×10⁶ SW620 cells on the right flank. Ten days after implant, and once tumors had reached size accurately measurable, mice were treated with 5-FU by itself, 5-FU combined with free d-Ino, or with the [5-FU+d-Ino]-L form as follows: 5-FU: 50 mg/kg, d-Ino, and 120 mg/kg. Drugs were administered intraperitoneously on a 3×/week basis for three consecutive weeks (e.g., D1/D2/D3, D8/D9/D10 and D16/D17/D18). Tumor size was measured three times a week in three dimensions using Vernier calipers, and tumor weight (mg) was calculated using the following standard formula: mass=pi/6XlengthXwidthXheight (Waterhouse et al, 2005). Preliminary experiments performed with empty liposomes were conducted to confirm the absence of any in vivo antiproliferative activity. Animal weight was monitored as a marker of toxicity. Animals were euthanized whenever a 25% loss of initial weight was observed, or when tumors reached 2500 mg.

### Results

### Encapsulation Rate and Release Studies

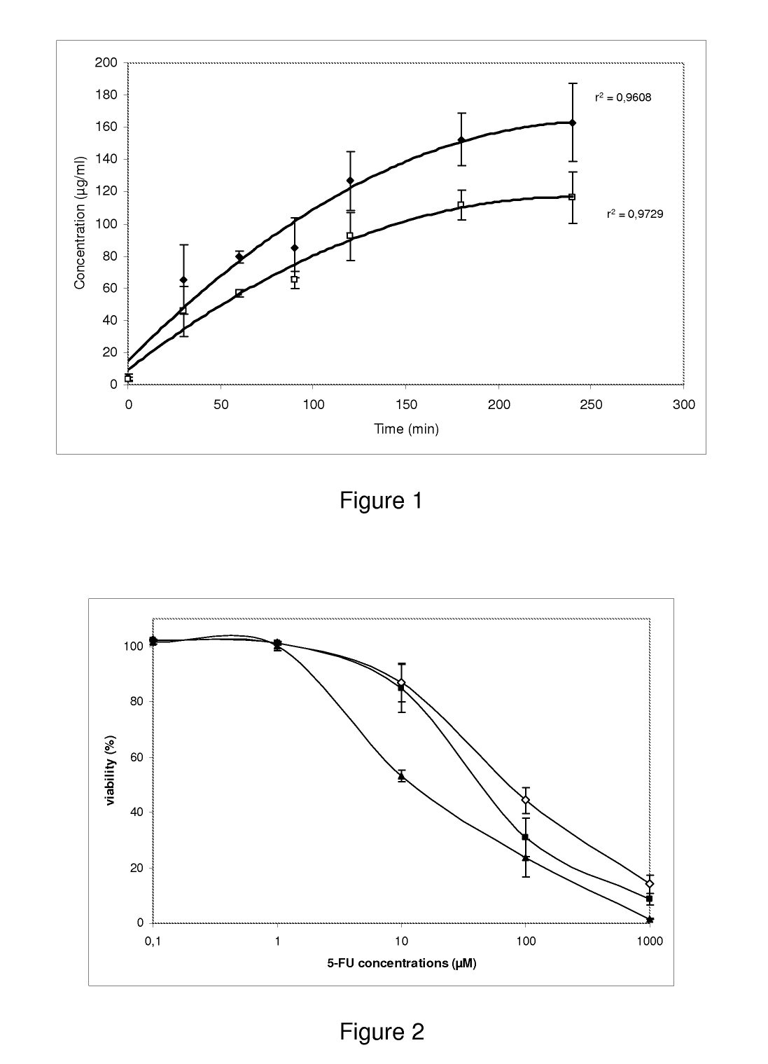

Homogenous, 100 nm-diameter liposome populations were obtained. Encapsulation rates of 5-FU and d-Ino were 10.6±1.6% and 26.2±5.3%, respectively. Release curves for 5-FU and d-Ino had similar profiles (n=5). Both were described by a polynomial equation (FIG. 1). No significant difference was observed between the 180 and 240 min concentrations (p>0.05, t test). Maximum, 100% release from the liposomes was reached after 4-h incubation for both drugs.

### Modulation of Antiproliferative Activity

Empty liposomes showed no in vitro cytotoxicity (data not shown). Results of cytotoxic studies are summarized in FIG. 2. Combining 5-FU with either free d-Ino or used as a liposomal [5-FU+d-Ino]-L formulation led to a significant increase in cell sensitivity. Respective IC₅₀'s for 5-FU alone, freely associated with d-Ino, or encapsulated with d-Ino in a single liposome were 77±6, 57±13 and 48±6.4 μM, respectively (n=3). At the IC₅₀ level, use of free d-Ino caused a 26% improvement in 5-FU efficacy, whereas the double-agent liposomal formulation caused a 37% increase in SW620 sensitivity. Similarly, cell response was further improved by 52 and 77% (free d-Ino and liposomal formulation, respectively) at IC₂₀, and by 18 and 80% (free d-Ino/liposomes) at the IC₈₀ levels.

### Modulation of 5-FU Intracellular Activation

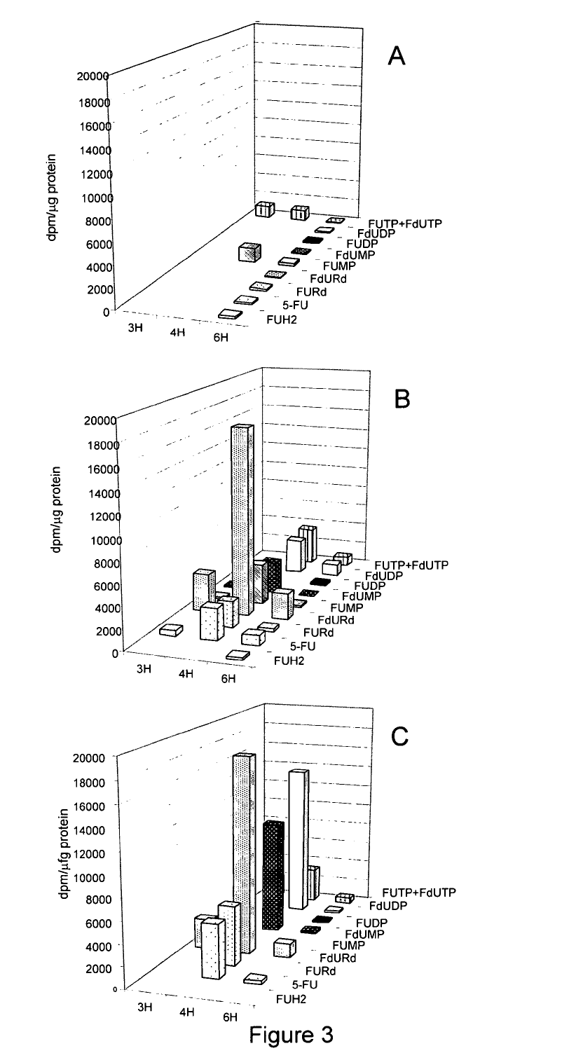

Intratumoral metabolic profiles of 5-FU used alone, combined with d-Ino or used as a liposomal formulation are displayed in FIG. 3. When used alone, 5-FU anabolism took place via the RNA pathway, and little or no FdUMP was formed over the 3-6 h observation period. Combining d-Ino with 5-FU led to a striking change in the activation pattern of 5-FU, with activation occurring predominantly through the DNA pathway, resulting in subsequent intracellular accumulation of fluoro-deoxynucleotides. Overall, anti-TS FdUMP synthesis was increased from 83 dpm/mg protein (5-FU alone) to 3199 dpm/mg (5-FU+d-Ino, +3801%) and to 11276 dpm/mg (liposomal combination, +13561%). When considering the total cytosolic amount of unchanged 5-FU and metabolites formed, data showed that exposing SW620 cells to the [5-FU+d-Ino]-L combination led to a 36% increase of drug uptake as compared with the free form combination.

### TS Inhibition Study

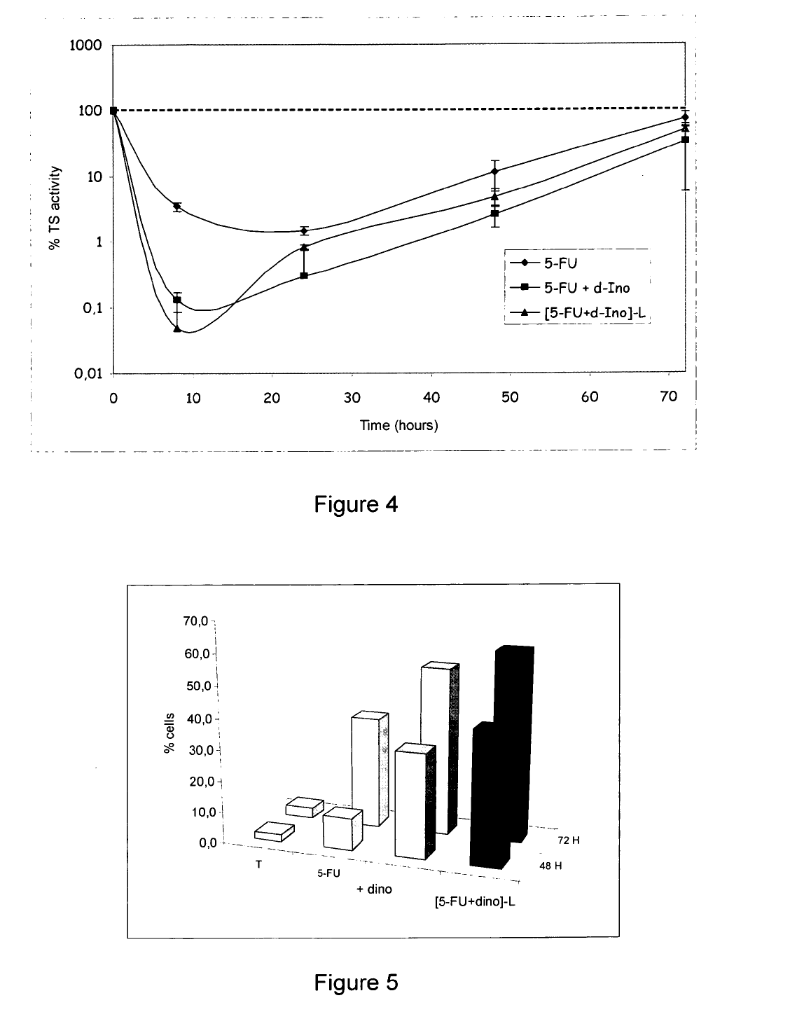

A significant improvement in TS inhibition was observed both with free d-Ino and with the encapsulated formulation (FIG. 4). TS activity was diminished by 96% after 8-h in cells exposed to 5-FU+d-Ino as compared to 5-FU alone. The liposomal formulation further improved this inhibition level by 61%, with an eventual 98% decrease in TS activity (n=3).

### Cell Cycle Analysis

Monitoring of the sub-G0/G1 population at 48 h after PI staining is displayed in FIG. 5. Results revealed a 324% higher induction of cell death by 5-FU when associated with free d-Ino, and a 408% increase with the liposomal form, as compared with 5-FU alone. At 72 h, increases in cell death of 150% and 169%, respectively with d-Ino or liposomal formulations (n=3) were observed.

### Apoptosis Studies

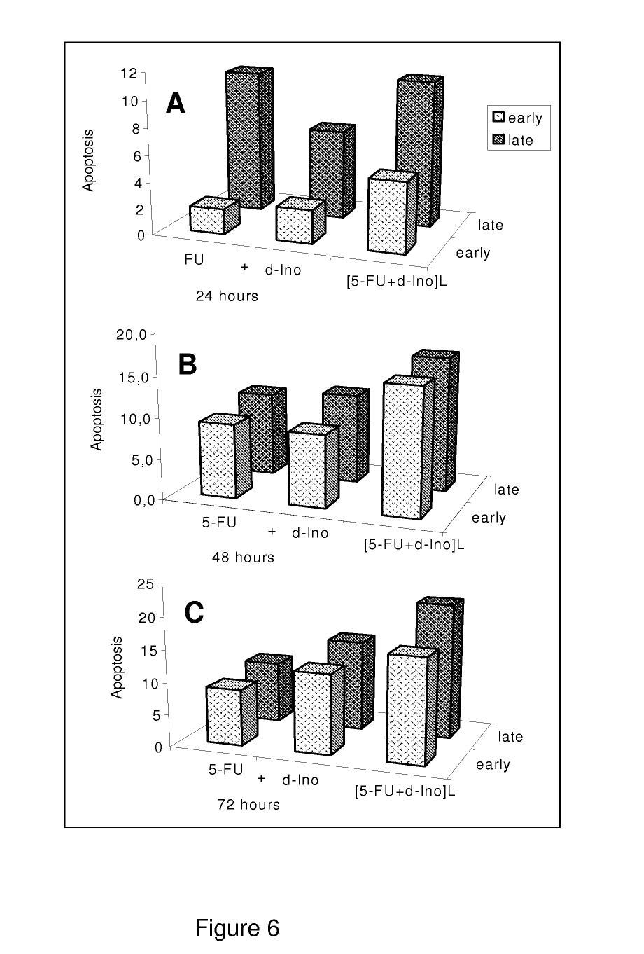

A greater induction of both early and late apoptosis was observed in SW620 exposed to FU modulated with free d-Ino, or the co-encapsulated form (FIG. 6). Early apoptosis induction was increased by 235, 103 and 136% at 24, 48 and 72 h respectively (free d-Ino) and by 326, 268 and 219% with the liposomal form, as compared with 5-FU alone. Similarly, late apoptosis was increased by 92, 119 and 138% after 24, 48 and 72 h (free d-Ino), and by 159, 206 and 219% with the encapsulated form, as compared with the use of standard 5-FU (n=4).

### Drug Monitoring Study.

Monitoring of 5-FU and d-Ino in plasma was performed after administration of these both drugs, injected either free or as a liposomal combination. Due to analytical interferences with merging endogenous peaks, 5-FU concentrations remained below our limit of detection, regardless of the formulation used. Conversely, d-Ino was fully measurable over the 60-120 min period and showed circulating concentrations up to 139% higher when administered as liposomes, as compared with the free form. At 120 min, 470 ng/ml of d-Ino was still measured in rats of the liposome group, whereas no modulator was detected anymore in animals treated with the free 5-FU+d-Ino association.

### In Vivo Efficacy Studies.

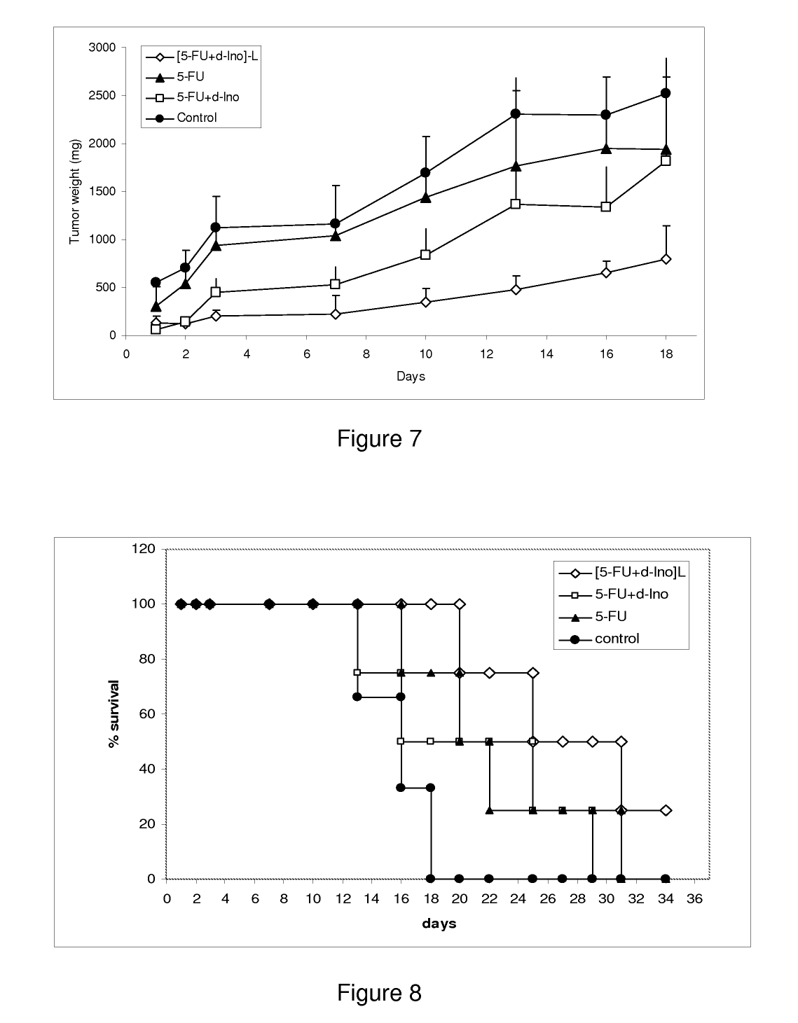

Treatment with the empty liposomes showed no impact on tumor growth as compared to untreated animals (data not shown). At the conclusion of the study, tumor size was reduced by 28% (n.$), 23% (n.$) and 74% (p<0.05) in mice treated with 5-FU alone, 5-FU with free d-Ino, or the [5-FU+d-Ino]-L formulation, respectively (FIG. 7). No signs of toxicity were observed in these animals, regardless of the treatment modality, and no statistical differences were found in animal weights among the different groups (data not shown). When compared to controls, survival time was increased by 25% in the 5-FU-treatment group (20 days vs 16 days), 56% in the group treated with the 5-FU+d-Ino combination, and by 94% in animals treated with the liposomal formulation (p<0.05, FIG. 8).

These results showed that it was possible to reproducibly co-encapsulate both 5-FU and its modulator, with encapsulation rates and release profiles comparable to the pharmacodynamics of these compounds, showing that intratumoral formation of active FdUMP in the 3-6H time window was associated with a maximum efficacy in digestive cancer models (Ciccolini et al, 2000b and 2001). However, co-encapsulation rates of both 5-FU and its modulator were relatively poor (11 and 26%, respectively). Such a moderate encapsulation rate for 5-FU is not surprising when considering the polar and amphoterous properties of this drug, that render its handling quite difficult when preparing standard pegylated-liposomes, as used here (Nii et al, 2005). Indeed, the present strategy was to develop a stealth delivery system as basic and as simple as possible, to easily standardize a fabrication process that could be performed in most laboratories equipped with standard apparatus and reagents.

In the current study, encapsulation rates proved to be highly reproducible throughout time (e.g., <2% for 5-FU), thus suggesting little batch-to-batch variation, likely to bias subsequent experiments. In vitro, reversal of the resistant-profile of our model was achieved, with increases in both cell death and apoptosis induction, as well as marked increases in sensitization (e.g., +80% at IC₂₀) of the SW620 cells to 5-FU. Further experiments confirmed that this increase in efficacy was due to a remarkable switch from the RNA to the DNA activation pathway, with increased formation of active FdUMP when 5-FU was modulated with d-Ino. Subsequent examination of TS activity, as a pharmacological endpoint, showed profound and sustained inhibition of this target, in cells exposed to the liposomal combination. Additionally, the inventors observed that besides the preferential activation towards FdUMP, a nearly 40% increase in overall 5-FU cytosolic levels was achieved in cells treated with the liposomal formulation, thus probably adding to the higher cytotoxicity effect that was subsequently measured. Interestingly, similar reversal of resistance to 5-FU was also achieved in mice. When used as monotherapy, 5-FU failed to reduce tumor growth as compared with untreated animals. It is noteworthy that combining 5-FU with free d-Ino hardly improved efficacy in this animal study, probably due to the relatively low doses of modulator used (120 mg/kg) and considering d-Ino's dramatic catabolism and the dosage that is normally required (3.2 g/kg) to achieve modulating effects in vivo (Ciccolini et al, 2000b and 2001). Conversely, [FU+d-Ino]-L caused a 69% reduction in tumor size when compared with untreated animals, and a significant 57% reduction when compared with 5-FU alone. Of note, this increase in efficacy was not accompanied with extra-toxicities and all animals showed excellent tolerance, thus indicating an obvious improvement in the therapeutic index of 5-FU. In concordance with the increased antitumoral efficacy and good tolerance, median survival time was nearly doubled in animals treated with our liposomal formulation, compared with standard 5-FU, thus demonstrating that chemoresistance to 5-FU could indeed be overcome.

### Example 2

### Alternative Composition

### Drugs and Chemicals

Egg yolk phosphatidylcholine (PC), phosphatidylglycerol (PG), cholesterol (C), polyethylene glycol (PEG) covalently binded to phosphatidylethanolamine, d-Ino, and 5-FU were all purchased from Sigma (St Quentin France).

Methods for preparation of Small Unilamellar Liposomes

Double-Liposomes ([5-FU+d-Ino]-L) were prepared by the classic thin film method (Olson et al, 1979). A lipid mixture composed of DSPC/C/PG/PEG 40:33:0.68:26 relative molar ratio was prepared.

Lipids were dissolved in methanol solution and subsequently dried in a round bottom flask to form a dried thin film and placed during 3 hours in a vacuum pump to remove solvent. The resulting lipid film was placed under high vacuum for a minimum of 2 hours. The lipid film was hydrated with carbonate buffer ph 7.4 containing the solution of 5-fluorouracil (0.2 mM) and 2′-deoxyinosine (0.24 mM) during 30 minutes at 37-40° C. Multilamellar vesicles (MLVs) were formed by vortex mixing the lipid dispersions at room temperature. After hydratation Small unilamellar vesivles (SUVs) were obtained by sonication with a sonic probe (20 kHz) for 5 min at 4° C. on ice.

The size distribution of liposomes obtained was homogenous and about 100 nm diameter. Non-encapsulated drugs were removed by centrifugation (10 000 g) using 30 000 kDa columns (e.g., Vivaspin®, Sartorius) for about 2 h. The fraction containing the liposomal drugs was resuspended next in either 10 ml of carbonate buffer or culture media, depending on the future use (in vitro, in vivo) of the preparation. Sterile liposome were obtained after extrusion through PVDF filters (0.22 μm pore size).

### Polydispersity Study

Distribution of liposome size was determined by dynamic light scattering with a submicron particle analyser (Sematech, Nice, France). Measurements were performed from a 90° angle at room temperature. A homogenous, 100 nm-diameter liposome's population was obtained. Encapsulation rates of 5-FU and d-Ino were 10.6±1.6% and 26.2±5.3%, respectively.

### Example 3

### Encapsulation of 5-FU, d-Ino, and Folinic Acid

### Drugs and Chemicals

Egg yolk phosphatidylcholine (PC), phosphatidylglycerol (PG), cholesterol (C), polyethylene glycol (PEG) covalently binded to phosphatidylethanolamine, d-Ino, and 5-FU were all purchased from Sigma (St Quentin France). Folinic acid was purchased from Dakota Pharm (Paris, France).

### Methods for Preparation of Small Unilamellar Liposomes

Triple-Liposomes ([5-FU+d-Ino+folinic acid]-L) were prepared by the classic thin film method (Olson et al, 1979). A lipid mixture composed of DSPC/PG/C/PEG 26/16.7/21/0.44 relative molar ratio was prepared.

Lipids were dissolved in methanol solution and subsequently dried under nitrogen flow, to form a dried thin film. The resulting lipid film was hydrated with carbonate buffer, and contacted to solution of 5-fluorouracil (0.2 mM) and 2′-deoxyinosine (0.24 mM) and folinic acid (0.2 mM) for encapsulation, during 30 minutes at 37° C. Small unilamellar vesivles (SUVs) were homogenized by sonication with a sonic probe (20 kHz) and centrifuged (9,000 g). The pellet is collected in an isotonic solution and ready to use.

### Encapsulation Rate and Release Studies

Homogenous, 100 nm-diameter liposome populations were obtained. Encapsulation rates of 5-FU, d-Ino and folinic acid were 10%, 21%, and 10% respectively. Maximum, 100% release from the liposomes was reached after 4-h incubation for the three drugs.

### Modulation of Antiproliferative Activity

Antitumoral efficacy of the triple liposomal formulation (5-FU+d-Ino+folinic acid) was evaluated in vitro on the 5-FU-resistant SW620 human colorectal cell line. Cell sensitivity was estimated by calculating the respective IC50 of the different regimen (MTT testing, 72 continuous exposure). Measured IC50 were 62 μM (5-FU alone), 21 μM (5-FU+folinic acid, a.k.a. Fufol protocol) and 5 μM (liposomal formulation). Liposomal formulation led to a maximum gain in efficacy of ×12 and ×4 vs standard 5-FU and standard Fufol.

### REFERENCES

Alley M C, Scudiero D A, Monks A, Hursey M L, Czerwinski M J, Fine D L, Abbott B J, Mayo J G, Shoemaker R H, Boyd M R (1988) Feasability of drug screening with panels of human tumor cell lines using a microculture tetrazolium assay. Cancer Res 48: 589-601.
Bangham, et al. J. Mol. Biol. (1965) 12: 238
Blanquicett C, Saif M W, Buchsbaum D J, Eloubeidi M, Vickers S M, Chhieng D C, Carpenter M D, Sellers J C, Russo S, Diasio R B, Johnson M R (2005) Antitumor efficacy of capecitabine and celecoxib in irradiated and lead-shielded, contralateral human BxPC-3 pancreatic cancer xenografts: clinical implications of abscopal effects. Clin Cancer Res 11:8773-81.
Boyer J, McLean E G, Aroori S, Wilson P, McCulla A, Carey P D, Longley D B, Johnston P G (2004) Characterization of p53 wild-type and null isogenic colorectal cancer cell lines resistant to 5-fluorouracil, oxaliplatin, and irinotecan. Clin Cancer Res 10: 2158-2167.
Chazal M, Cheradame S, Formento J L, Francoual M, Formento P, Etienne M C, Francois E, Richelme H, Mousseau M, Letoublon C, Pezet D, Cure H, Seitz J F, Milano G (1997) Decreased folylpolyglutamate synthetase activity in tumors resistant to fluorouracil-folinic acid treatment:clinical data. Clin Cancer Res 3: 553-557.
Ciccolini J, Peillard L, Aubert C, Formento P, Milano G, Catalin J (2000a) Monitoring of the intracellular activation of 5-fluorouracil to deoxyribonucleotides in HT29 human colon cell line: application to modulation of metabolism and cytotoxicity study. Fundam Clin Pharmacol 14: 147-54.
Ciccolini J, Peillard L, Evrard A, Cuq P, Aubert C, Pelegrin A, Formento P, Milano G, Catalin J (2000b) Enhanced antitumor activity of 5-fluorouracil in combination with 2′-deoxyinosine in human colorectal cell lines and human colon tumor xenografts. Clin Cancer Res 6: 1529-35.
Ciccolini J, Cuq P, Evrard A, Giacometti S, Pelegrin A, Aubert C, Cano J P, Iliadis A (2001) Combination of thymidine phosphorylase gene transfer and deoxyinosine treatment greatly enhances 5-fluorouracil antitumor activity in vitro and in vivo. Mol Cancer Ther 1: 133-9.
Ciccolini J, Evrard A, Cuq P (2004) Thymidine phosphorylase and fluoropyrimidines efficacy: a Jekyll and Hyde story. Curr Med Chem Anticancer Agents 4: 71-81.
Deamer and Bangham, Biochiln. Biopl vs. Acta (1976) 443: 629
De Bruin M, van Capel T, Smid K, van der Born K, Fukushima M, Hoekman K, Pinedo H M, Peters G J (2004) Role of platelet derived endothelial cell growth factor/thymidine phosphorylase in fluoropyrimidine sensitivity and potential role of deoxyribose-1-phosphate. Nucleosides Nucleotides Nucleic Acids 23: 1485-90.
Derzynkiewicz Z, Bruno S, Del Bino G, Gorczyca W, Hotz M A, Lassota P, Traganos F (1992) Features of apoptotic cells measured by flow cytometry. Cytometry 13: 95-808.
Diasio R B, Harris B E (1989) Clinical pharmacology of 5-fluorouracil. Clin Pharmacokinet 16: 215-237.
Evrard A, Cuq P, Ciccolini J, Vian L, Cano J P (1999) Increased cytotoxicity and bystander effect of 5-fluorouracil and 5-deoxy-5-fluorouridine in human colorectal cancer cells transfected with thymidine phosphorylase. Br J Cancer 80: 1726-33.
Fanciullino R, Giacometti S, Aubert C, Fina F, Martin P M, Piccerelle P, Ciccolini J (2005) Development of stealth liposome formulation of 2′-deoxyinosine as 5-fluorouracil modulator: in vitro and in vivo study. Pharm Res 22: 2051-7.
Fanciullino R, Evrard A, Cuq P, Giacometti S, Peillard L, Mercier C, Aubert C, Milano G, Ciccolini J (2006) Genetic and biochemical modulation of 5-fluorouracil through the overexpression of thymidine kinase: an in-vitro study. Anticancer Drugs 17:463-70.
Johnston P G, Lenz H J, Leichman C G, Danenberg K D, Allegra C J, Danenberg P V, Leichman L (1995) Thymidylate synthase gene and protein expression correlate and are associated with response to 5-fluorouracil in human colorectal and gastric tumors. Cancer Res 55: 1407-1412.
Langley D B, Harkin D P, Johnston P G (2003), 5-fluorouracil mechanisms of action and clinical strategies. Nat Rev Cancer 3: 330-338.
Nii T, Ishii F (2005) Encapsulation efficiency of water-soluble and insoluble drugs in liposomes prepared by the microencapsulation vesicle method. Int J Pharm 298: 198-205.
Olson F, Hunt C A, Szoka F C, Vail W J, Papahadjopoulos D (1979) Biochim Biophys Acta 557: 9-23.
Peters G J, Backus H H, Freemantle S, van Triest B, Codacci-Pisanelli G, van der Wilt C L, Smid K, Lunec J, Calvert A H, Marsh H, McLeod H L, Bloemena E, Meijer s, Jansen G, van Groeningen C J, Pinedo H M (2002) Induction of thymidylate synthase as a 5-fluorouracil resistance mechanism. Biochim Biophys Acta 1587: 194-205.
Pinedo H M, Peters G J (1988) Fluorouracil: biochemistry and pharmacology. J Clin Oncol 6: 1653-1664.
Roberts D (1966) An isotopic assay for thymidylate synthetase. Biochemistry 5: 3546-3548.
Schwartz E L, Baptiste N, Megati S, Wadler S, Otter B A (1995a) 5-Ethoxy-2′-deoxyuridine, a novel substrate for thymidine phosphorylase, potentiates the antitumor activity of 5-fluorouracil when used in combination with interferon, an inducer of thymidine phosphorylase expression. Cancer Res 55: 3543-50.
Schwartz E L, Baptiste N, Wadler S, Makower D (1995b) Thymidine phosphorylase mediates the sensitivity of human colon carcinoma cells to 5-fluorouracil. J Biol Chem 270: 19073-7.
Szoka and Paphadjopoulos, P. N. A. S. (1978) 75: 4194.
Waterhouse D N, Madden T D, Cullis P R, Bally M B, Mayer L D, Webb M S (2005) Preparation, characterization, and biological analysis of liposomal formulations of vincristine. Methods Enzymol 391: 40-57.

1. A pharmaceutical composition comprising liposomes which coencapsulate 5-fluorouracil (5-FU) and 2′-deoxyinosine (d-Ino).

2. The pharmaceutical composition of claim 1, wherein the liposomes comprise phosphatidylcholine (PC), phosphatidylglycol (PG) and sterol.

3. The pharmaceutical composition of claim 2, wherein the liposomes comprise phosphatidylcholine (PC), phosphatidylglycol (PG) and sterol in a molar ratio PC/PG/sterol of 20-80/10-60/15-60.

4. The composition of claim 3, wherein the liposomes comprise a molar ratio PC/PG/sterol of 30-50/20-30/20-40.

5. The composition of claim 4, wherein the liposomes comprise a molar ratio PC/PG/sterol of 35-45/24-28/24-38.

6. The composition of claim 2, wherein the sterol is cholesterol.

7. The composition of claim 2, further comprising polyethylene glycol (PEG) out of the liposome membrane.

8. The composition of claim 7, wherein the liposomes comprise phosphatidylcholine (PC), phosphatidylglycol (PG), sterol and PEG in a molar ratio PC/PG/sterol/PEG of 20-80/10-60/15-60/0.30-1.5.

9. The composition of claim 8, wherein the liposomes comprise a molar ratio of PC/PG/cholesterol/PEG of about 40/26/33/0.68.

10. The composition of claim 1, wherein the liposomes have a mean diameter of between 50 and 200 nm.

11. The composition of claim 1, wherein the liposomes further encapsulate folinic acid.

12. A method for preparing liposomes which coencapsulate 5-fluorouracil (5-FU) and 2′-deoxyinosine (d-Ino), which method comprises the steps consisting of
(a) dissolving a lipid mixture comprising phosphatidylcholine (PC), phosphatidylglycol (PG) and sterol in an alcoholic solvent, and removing the solvent to form a lipid film;
(b) hydrating the lipid film with a solution containing 5-fluorouracil and 2′-deoxyinosine, to form a lipid dispersion;
(c) forming Multilamellar vesicles (MLVs) by mixing the lipid dispersion;
(d) obtaining Small unilamellar vesicles (SUVs) therefrom, by sonication.

13. The method of claim 12, wherein the solution of step (b) further comprises folinic acid, whereby liposomes which coencapsulate 5-FU, d-Ino, and folinic acid, are prepared.

14. Liposomes obtainable by the method of claim 12.

15. A method for treating cancer in a patient, which method comprises administering an effective amount of a pharmaceutical composition comprising liposomes which coencapsulate 5-fluorouracil (5-FU) and 2′-deoxyinosine (d-Ino) in a patient in need thereof.

16. The method of claim 15, wherein the patient has shown chemoresistance to 5-FU or to another fluoropyrimidine drug.

17. The composition of claim 3, wherein the sterol is cholesterol.

18. The composition of claim 4, wherein the sterol is cholesterol.

19. The composition of claim 5, wherein the sterol is cholesterol.

20. The composition of claim 3, further comprising polyethylene glycol (PEG) out of the liposome membrane.
